(19) United States
(12) Patent Application Publication
(10) Pub. No.: US 2022/0280038 A1
(43) Pub. Date: Sep. 8, 2022

(54) Circadian Intraocular Pressure Profile and Methods of Treatment

(71) Applicant: InjectSense, Inc., Emeryville, CA (US)

(72) Inventors: Ariel Cao, Emeryville, CA (US); James Ball, Minneapolis, MN (US); Sebnem Acar, Emeryville, CA (US); Thomas H. Breunig, Emeryville, CA (US)

(73) Assignee: InjectSense, Inc., Emeryville, CA (US)

(21) Appl. No.: 17/688,408

(22) Filed: Mar. 7, 2022

Related U.S. Application Data
(60) Provisional application No. 63/158,073, filed on Mar. 8, 2021.

(51) Int. Cl. A61B 3/16; G16H 50/20; G16H 50/30

(52) U.S. Cl. CPC A61B 3/16; G16H 50/20; G16H 50/30

(57) ABSTRACT

Systems and methods of assessing and treating eye disease. Such systems and methods can include presenting intraocular pressure (IOP) data as a circadian IOP profile on a polar plot over a 24-hr period to facilitate identification of cyclical variations of IOP occurring on a daily basis. Such methods can further include associating a particular circadian IOP profile with structural and/or a functional assessments, and utilizing such associations from multiple patients to facilitate identification of relationships to provide improved assessment and treatment of eye disease.

### CROSS-REFERENCES TO RELATED APPLICATIONS

The present application claims the benefit under 35 USC § 119(e) of U.S. Provisional Appln. No. 63/158,073 filed Mar. 8, 2021; the full disclosure which is incorporated herein by reference in its entirety for all purposes.

The present application is generally related to the following co-assigned applications: U.S. Pat. No. 10,213,107 entitled “Methods and Devices for Implantation of Intraocular Pressure Sensors”; U.S. Publn. No. 2016/0000344 entitled “Hermetically Sealed Implant Sensors with Vertical Stacking Architecture”; U.S. Publn. No. 2016/0058324 entitled “Ultra Low Power Charging Implant Sensors with Wireless Interface for Patient Monitoring”; and PCT Publn No. WO 2021/003434 entitled “Hermetic Heterogeneous Integration Platform for Active and Passive Electronic Components”; all of which are incorporated herein by reference in their entirety for all purposes.

### FIELD OF THE INVENTION

The invention pertains to improved monitoring of intraocular pressure (IOP) for diagnostics and treatment of eye disease.

### BACKGROUND OF THE INVENTION

The current standard in intraocular pressure (IOP) monitoring is to represent IOP data by a linear x-y graph (e.g., time versus IOP) based on limited discrete data points (rarely for more than a few days and typically based on one measurement or a few measurements per day), such as shown in FIGS. 6A-6B. Even when a few samples are taken per day, nocturnal periods are not typically collected since the cornea is not readily accessed during sleep. The risk assessment focus of conventional treatment approaches is to assess IOP average reduction against a baseline (pre-medication) and actual data collected, typically, one sample or a few samples per day.

The polygonal chain representation seen in FIGS. 6A-6B is an incomplete representation of information regarding therapy effectiveness (limited time window) and even fluctuation is not adequately captured since spacing between sampling is still significant. It is understood that IOP daily can fluctuate as much as 6 mmHg during the day. Accordingly, periods of elevated IOP associated with glaucoma can be difficult to identify and treat, which leads to gradual worsening of disease state and progressive loss of vision.

Therefore, there exists a need for an approach that allows for improved monitoring of IOP and assessments to provide improved risk assessment and treatment of patients having elevated IOP associated with glaucoma.

### BRIEF SUMMARY

In one aspect, the present invention pertains to presenting IOP data as a circadian IOP profile on a polar plot of a 24-hr period to facilitate identification of cyclical variations of IOP data that occur on a daily basis. These variations can be associated with structural and functional assessments to facilitate improved diagnosis and treatment of patients having elevated IOP associated with eye disease.

In one aspect, the method pertains to a method of diagnosing and/or treating an eye of a select patient, that includes steps of: receiving a first plurality of intraocular pressure (IOP) measurements over a first 24-hour period from the select patient and determining a circadian IOP profile in a polar format based on the plurality of IOP measurements. The method can further include receiving a second plurality of intraocular pressure measurements over a second 24-hour period such that the circadian IOP profile is based on both the first and second plurality of IOP measurements (e.g., average between multiple days). In some embodiments, the method entails displaying the circadian IOP profile on a graphical user interface along with a targeted IOP band of suitable IOPs between an upper and lower limit. The circadian IOP profile can also be displayed along with a baseline IOP profile for ease of comparison. The method can receive the IOP measurements from an implantable IOP sensor device having an IOP sensor portion that is disposed entirely within the vitreous body of the eye of the select patient or any suitable IOP sensor.

Such methods can further include: determining a pattern of exceedances of the circadian IOP profile from the target IOP range (e.g., a number, quantity, timing, duration of exceedances, or any combination thereof). The method can then compare the circadian IOP profile of the select patient with a plurality of circadian IOP profiles of a first set of patients; and determine a risk assessment of the select patient and/or a course of treatment based on the comparison. In some embodiments, the method compares the pattern of IOP exceedances for the select patient with a library of patterns from one or more sets of patients. In some embodiments, the baseline IOP between patients is also accounted for.

Methods of treatment can further include: receiving a second plurality of IOP measurements from the select patient during treatment; determining an updated circadian IOP profile from the second plurality of IOP measurement; determining an updated pattern of IOP exceedances from the updated circadian IOP profile; and determining an updated risk assessment and/or course of treatment based on the comparison.

In another aspect, methods of diagnosis and/or treatment can include: obtaining a circadian IOP profile from multiple patients that are associated with one or more of a structural assessment and a functional assessment to determine a relationship or associated between IOP profile and structural and/or functional damage. The method can further include obtaining a circadian IOP profile from a select patient and determining one or more attributes of the IOP profile and determining a risk assessment for structural and/or functional damage based on the determined relationship. In some embodiments, this relationship is defined within a library or lookup table.

In yet another aspect, the invention pertains to a system for diagnosis and/or treatment of an eye of a select patient, the system including a graphical user interface; and a processor having a memory with programmable instructions recorded thereon, the instructions configured to: receive a first plurality of intraocular pressure (IOP) measurements over a first 24-hour period from the select patient and output to the graphical user interface, a circadian IOP profile in a polar format based on the plurality of IOP measurements. The system can further be configured to determine a pattern of exceedances of the circadian IOP profile from a suitable range of IOP (e.g., a number, quantity, timing, or duration of exceedances). The system can then compare the circadian IOP profile of the select patient with multiple circadian IOP profiles from a first set of patients; and determine a risk assessment of the select patient and/or a course of treatment based on the comparison. In some embodiments, the method compares a pattern of IOP exceedances for the select patient with a library of exceedance patterns from a first set of patients. The system can further include an implantable IOP sensor device having an IOP sensor portion that is disposed entirely within the vitreous body of the eye of the select patient or any suitable IOP sensor.

Other features and advantages of the invention shall be apparent based upon the accompanying description, drawings, and claims.

### BRIEF DESCRIPTION OF THE DRAWINGS

FIG. 1 depicts a graphical user display depicting circadian intraocular pressure data along with a target IOP profile, which is a range of acceptable IOP, in accordance with some embodiments.

FIG. 2 depicts a graphical user display depicting circadian intraocular pressure data for left and right eyes along with the target IOP profile, in accordance with some embodiments.

FIG. 3 depicts a graphical user display depicting a circadian IOP profile linked with a structural eye assessment, in accordance with some embodiments.

FIG. 4 depicts a graphical user display depicting a circadian IOP profile linked with a functional eye assessment, in accordance with some embodiments.

FIG. 5 depicts a graphical user display depicting a circadian IOP profile linked with both structural and functional eye assessments, in accordance with some embodiments.

FIGS. 6A-6B depict conventional linear graphs of IOP data in accordance with conventional methods of treatment and diagnosis.

FIG. 7 depicts a library of relationships between variations in IOP and structural and functional assessments identified from a set of patients, in accordance with some embodiments.

FIG. 8 depicts a method of diagnosis and/or treatment of an eye, in accordance with some embodiments.

FIG. 9 depicts another method of diagnosis and/or treatment of an eye, in accordance with some embodiments.

FIG. 10 depicts yet another method of diagnosis and/or treatment of an eye, in accordance with some embodiments.

FIG. 11 depicts a decision making process, in accordance with some embodiments.

### DETAILED DESCRIPTION

Although this disclosure is detailed enough to enable those skilled in the art to practice the invention, the embodiments herein disclose mere examples of the invention and may be embodied by varying approaches without departing from the scope and spirit of the

I. Circadian IOP Profile/Targeted IOP Profile (TIP)

In one aspect, the invention shifts the paradigm from considering discrete IOP data points to providing an IOP profile determined from sufficiently high frequency sampling of IOP so as to allow identification of regular or cyclical variations of IOP. In some embodiments, the IOP profile is a circadian profile, for example a profile of IOP data over a 24-hr period presented in a polar graph. In some embodiments, the IOP profile provides a higher accuracy (+/−1 mmHg) with an IOP dataset of 24 hours increments (e.g., from minutes to hour sampling). Such an approach allows a physician to identify a circadian rhythm of IOP variations, and a range of patterns can be defined and identified. In some embodiments, such IOP profiles are obtained from multiple patients across one or more patient populations and compared with various other patient attributes and/or assessments such that broader relationships between patterns in IOP profiles and eye conditions can be ascertained. Such comparisons can be performed across relatively large numbers of patients through data-mining (e.g., AI and Deep learning) so as to allow identification of relationships that otherwise could not be determined.

In some embodiments, multiple IOP measurements are obtained from an implantable sensor implanted within the eye. Preferably, the sensor is implanted so that a sensor portion is implanted entirely within the vitreous body so as to obtain improved measurements of IOP, for example by the implantation approach described in U.S. Patent Application No. 62/019,826, which is incorporated herein by reference, or any suitable implantation approach. Such a sensor can include any of the configurations depicted in U.S. Patent Application No. 62/019,841, which is incorporated herein by reference, or any suitable sensor. Further, it is desirable for the IOP sensor to obtain high frequency sampling (e.g., from minutes to hour sampling) and facilitate telemetry of the obtained measurements, for example by any of the approaches described in U.S. Patent Application No. 62/044,895, which is incorporated herein by reference, or any suitable approach.

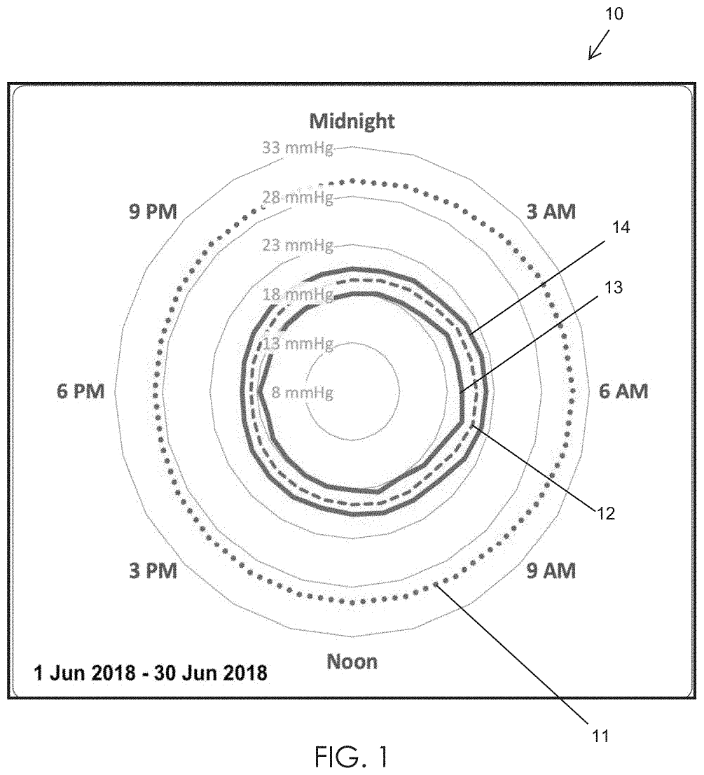

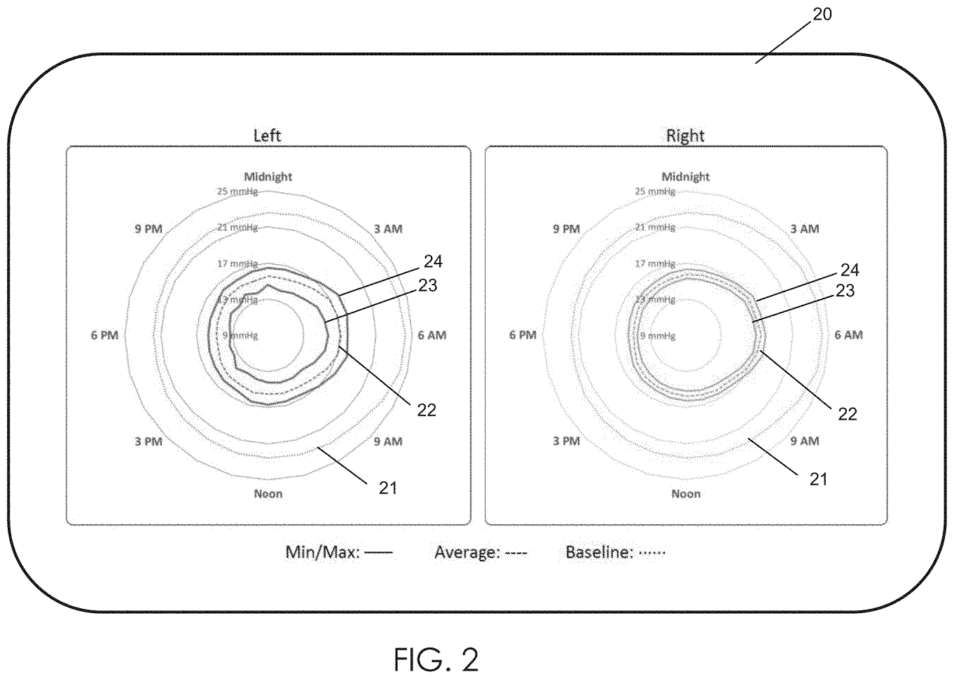

Such a presentation can be presented on a graphical user display of a treatment system, which can include any computing device (e.g., laptop, tablet, smartphone) of a clinician, to facilitate identification of variations of IOP due to cyclical patterns and/or circadian rhythm. While represented as a 24-hr period, this representation can aggregate IOP data collected from each day over longer periods of time (e.g., over an entire month). Such a presentation of IOP data allows the physician to identify cyclical trends that occur over a 24-hr period, which can be used to identify: a signature of non-compliance, dosage issues, multiple drugs combinations, drug interference, drug latency (e.g., pharmaco-kinetics) and also to define the effectiveness pattern of a specific drug specific to a specific patient (i.e., a personalized response). This is especially important to in managing therapy so as to adequately maintain IOP below the targeted IOP maximum value to prevent progression of the disease. This representation is disease agnostic and can also provide detailed metrics for more effectiveness comparisons. Examples of such circadian IOP profiles presented with a targeted IOP profile is shown in FIGS. 1 and 2.

This relationship can be graphically represented to the user as shown, or by various other graphical representations (e.g., a heat map indicating IOP exceedance by time of day). This understanding of cyclical variations of IOP data improves monitoring and management of IOP to achieve a targeted maximum IOP value to prevent progression of eye disease. The polar graph can further include additional parameters (e.g., oxygen, eye motion, ocular pulse amplitude, eyeball motion REM, etc.) to identify relationships between multiple factors.

FIG. 1 presents a single circadian IOP profile graphical user display 10, which can represent the IOP profile of one eye, or can represent a composite of both eyes within a single plot. In this embodiment, the circadian IOP profile is defined as a radial/circular graph displaying IOP on a 24-hour time scale. The hours of the day are labeled outside the circle in a clockwise direction starting with midnight at the top while IOP is indicated by the distance from the center of the circle. Data points closer to the center of the circle represent lower pressures while data points farthest from the center are the highest pressures (e.g., pressure gradations of 8, 13, 18, 23, 28 and 33 mm Hg). This depiction is intended to provide a simple summary of IOP over a long period of time, while still providing detailed information about the circadian aspect of IOP variability. In this embodiment, the circadian IOP profile 12 is shown as a dashed line and represents the average IOP each day during the month. Optionally, the plot also includes a baseline IOP profile 11, which allows for an easy comparison between treated and untreated IOP.

The Targeted IOP Profile (TIP) is defined as the band of suitable IOP that needs to be achieved by the therapy during any 24 period. In some embodiments, the circadian rhythm and presentation will also allow to overlap weeks, months and years of data but still allow the system to access any specific data point for specific time window of interest. In this embodiment, the TIP is defined as a target band of suitable IOPs that is defined between a lower IOP boundary 13 and an upper IOP boundary 14. Treatment aims to keep the IOP profile within the target band of acceptable IOP. The IOP profiles allows the physician to readily identify any variations in IOP that exceed the targeted IOP band as well as the duration of each exceedance and the total exceedance during a 24-hr period. In some embodiments, the multiple IOP profiles are superimposed on a single graph. In some patients, IOP pressures are fairly consistent between eyes such that the IOP pressures can be adequately represented by a single composite profile.

It is understood that this is but one example of a circadian IOP profile and that various other configurations could be realized, for example, multiple days could be plotted on a single graph (rather than max/min averages), or the various attributes of the graph can be adjustable by the user. The described approach of a circadian IOP profile allows a clinician quick determination of control over the indicated time period, comparisons to the baseline, ready visualization of the variation of IOP during the course of the day (e.g., day versus night), and simplifies display of a summary of long-term data. The polar diagram can also be used at different level of details and also define a framework to identify alerts (e.g., high-pressure excursion during surgery for acute, trauma on eyeball with impact, change of patient in the hemodynamic conditions with supine or prone positions, patients activities).

It is appreciated that various other parameters could be included with the circadian IOP profile as well. Other parameters might include dynamically monitoring like level of oxygen saturation, eye motion (REM during sleep), ocular pulse amplitude or OPA (for blood perfusion monitoring), cardiac conditions such as CAS (Carotid Artery Stenosis) which can be estimated using OPA amplitude reduction, or any attribute desired.

FIG. 2 shows a dual IOP profile display 20 depicting IOP profiles for each of the left and right eye of a patient. Each IOP profile includes a baseline IOP profile 21, a treatment IOP profile 22, and a TIP between lower boundary 22 and upper boundary 24. Such a display is particularly useful in a patients having a tendency toward differing IOPs between eyes, as shown in FIG. 2. In some patients, the IOP profiles can differ significantly between eyes such that one eye may require a different treatment regimen than the other eye (e.g., higher dosage or more frequent administration of pressure reducing eye drops).

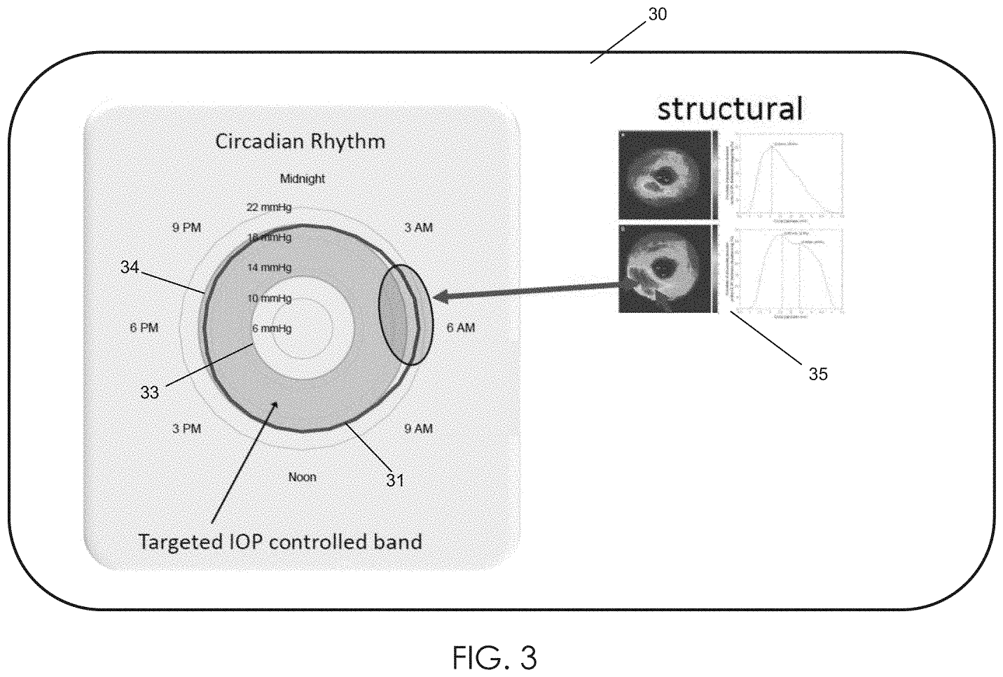

As shown above in FIGS. 3-5, the circadian IOP profiles can further be integrated with and/or linked with assessments of eye health inform, which further informs the diagnosis and/or treatment of patients by allowing comparison to patients with similar IOP profiles and/or assessments. For example, a particular circadian IOP profile can be associated with a structural assessment of the eye (e.g., optical coherence tomography (OCT)) or with a functional assessment of the eye (e.g., by Humphrey Visual Field (HVF) testing). The circadian IOP profiles can then be compared to repeated assessments over time to determine a disease progression associated with a particular IOP profile. This approach can be presented on a graphical user display, as shown in FIGS. 3-5, or otherwise incorporated into automated methods of diagnosis and/or treatment of a software treatment system. Such systems can utilize algorithms, artificial intelligence (AI) or deep learning (DL) to identify circadian patterns of IOP and their affect or relationship to structural and functional changes of the retina/optic nerve. Such relationships can greatly improve diagnostics, monitoring and treatment of eye disease.

II. Integrated TIP to RNFL/VF

In another aspect, the circadian IOP profile can be included within a data visualization model that allows through specific medical studies to cross-link IOP, RNFL (retinal fiber layer) structural changes and VF (visual field) functional change. Multiple studies have demonstrate that IOP levels directly affect the progression rate of glaucoma as initially structural changes will happen prior to functional/vision loss. This approach allows more complex relationships between IOP profiles and patterns in IOP variations and disease progression to be determined.

Due to the differences in data representation (e.g., time based IOP, imaging by OCT and HVF by perimetry), the analysis of such data may utilize advanced processing (e.g., advanced algorithms, AI, DL). The challenge, which falls into medical research, is to identify the pattern of IOP that will induce structural and functional changes within the retina/optic nerve. Examples of such data visualizations are shown below in FIGS. 3-5.

FIG. 3 depicts a graphical user display 30 in which a circadian IOP profile 31 is linked with a structural assessment (e.g., OCT) 35 of a patient. The circadian IOP profile 31 indicates when the IOP exceeds the targeted IOP band defined between lower boundary 33 and upper boundary 34. The structural assessment 35 indicates structural changes within the eye, which may indicate damage in the early stages of eye disease before the patient experiences noticeable vision loss, and may be used to monitor structural changes as the eye disease progresses. The structural changes may be linked and associated with attributes of the IOP profile (e.g., duration and/or magnitude of IOP exceedance). This approach can be used to demonstrate that patients with similar IOP profiles may experience similar structural damage and can be used in risk assessment as well as preventative treatments to halt progression of eye disease.

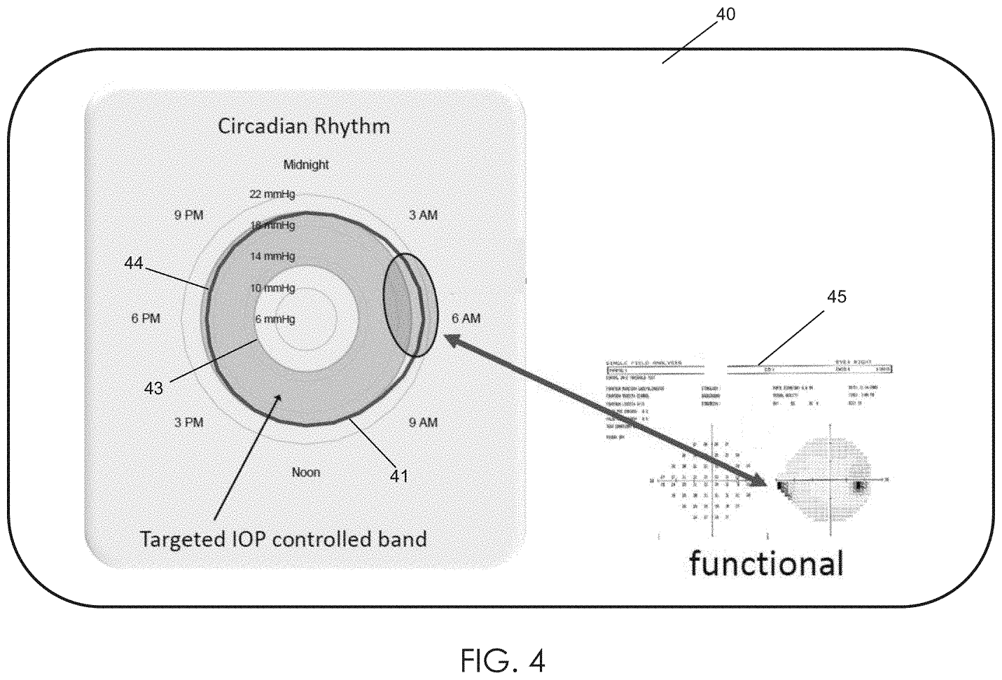

FIG. 4 depicts a graphical user display 40 in which a circadian IOP profile 41 is linked with a functional assessment (e.g., HVF) 45 of a patient. The circadian IOP profile 41 indicates when the IOP exceeds the targeted IOP band defined between lower boundary 43 and upper boundary 44. The functional assessment 45 indicates functional changes within the eye that indicate the extent of functional vision loss in a patient with progressively worsening state of eye disease. The functional changes may be linked and associated with attributes of the IOP profile (e.g., duration and/or magnitude of IOP exceedance). This approach can be used to demonstrate that patients with similar IOP profiles may experience similar functional damage and can be used in risk assessment as well as treatment to slow or halt further progression of eye damage.

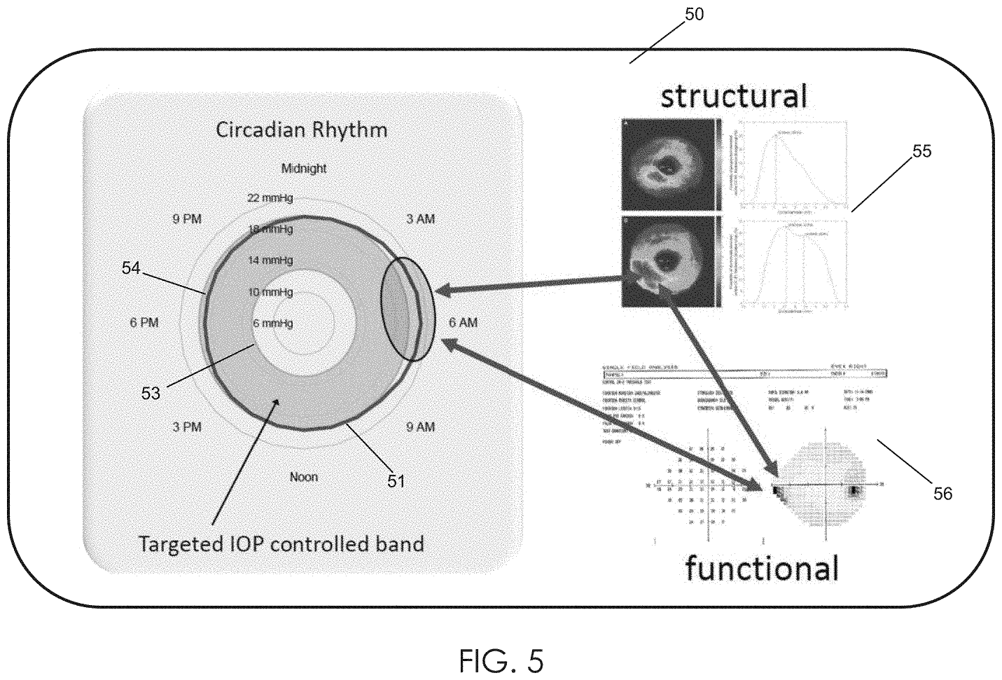

FIG. 5 depicts a graphical user display 50 in which a circadian IOP profile 51 is linked with a structural assessment (e.g., OCT) 55 and functional assessment (e.g., HVF) 56 of a patient. The circadian IOP profile 51 indicates when the IOP exceeds the targeted IOP band defined between lower boundary 53 and upper boundary 54. This association allows the structural damage and functional damage to be linked and associated with attributes of the IOP profile (e.g., duration and/or magnitude of IOP exceedance) and can be used to identify links between structural damage and functional damage in patients with similar IOP profiles. Similarly, this approach can be used to identify similar IOP variations in patients revealing similar structural and functional damage such that potential causes and treatments can be more readily identified.

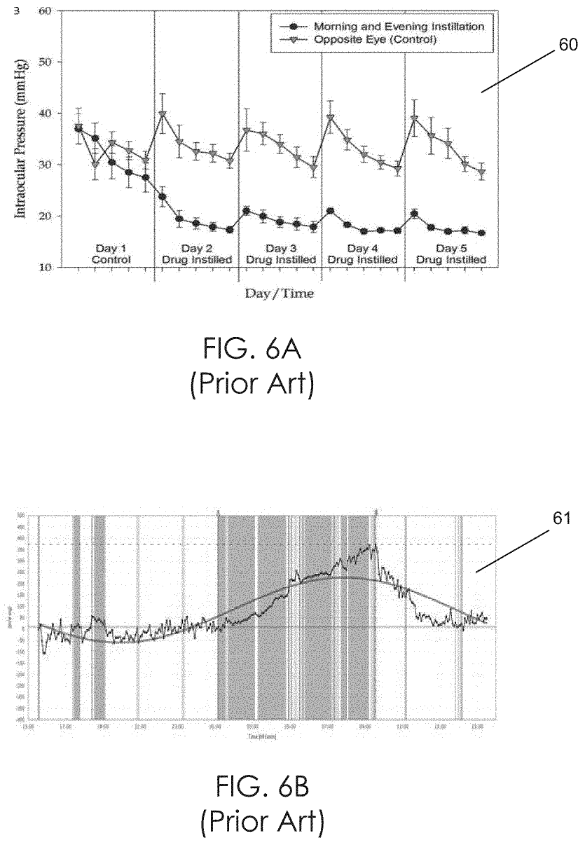

FIGS. 6A-6B illustrate conventional approaches to monitoring and plotting changes in IOP according to conventional treatment. As can be understood, by plotting variations based on discrete, infrequent sampling allow for only a low-resolution, general plotting of IOP data. Such an approach does not adequately capture the duration, magnitude and quantity of IOP exceedances that may cause significant damage. More importantly, this conventional approach is insufficient to identify cyclical variations that may occur repeatedly on a daily basis and cause considerable and repeated damage to a patient's eye. Even as little as an hour of elevated IOP each day, can cause damage to eye tissue such that over the course of years, a patient experiences disease progression and considerable vision loss. The present invention allows for identification of patterns that identify even relatively short IOP exceedances so that causal factors can be identified and addressed so as to slow or halt eye disease progression.

In another aspect, associating structural and functional assessment to circadian IOP profiles in multiple patients (e.g., hundreds of patients) allows for improved risk assessment in similar patients. Additionally, the IOP profiles can be further associated with one or more patients attributes (e.g., gender, age, baseline IOP, ethnicity, location, occupation, blood pressure, secondary disease, etc.) so as to further improve concordance between a select patient and relevant patient populations.

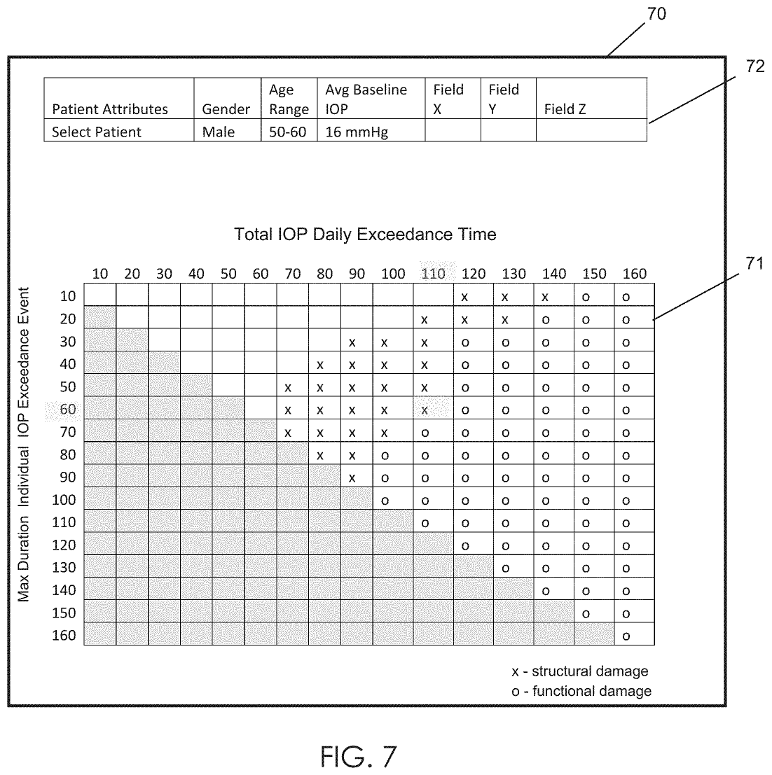

As shown in FIG. 7, the system can include a risk assessment 70 that can be used to link attributes of a select patient's circadian IOP profile with a potential for structural and/or functional damage based on similar patients. In this embodiment, a lookup table or library 71 is defined from multiple patients based on associations between circadian IOP profile attributes and structural and functional damage (such as that shown in FIG. 5). A circadian IOP profile can be determined for a select patient and attributes of the circadian IOP profile that may be associated with structural or functional eye damage are identified and used with the look-up table to determine a risk assessment based on associations of similar patients or patient populations.

In this embodiment, a circadian IOP profile of a select patient indicates a daily IOP exceedance of 110 minutes and a maximum duration of an individual exceedance event of 60 minutes. According to the lookup table based on associations of similar patients, these IOP profile attributes are associated with structural damage (x), which is on the verge of functional damage (o). In this embodiment, the association can further include a Patient Attribute Selection Menu 72 that allows the clinician to further refine the risk assessment by selecting attributes of the select patient to narrow the relevant patient population to similar patients. This approach can indicate the likelihood of structural and/or functional eye damage or a worsening disease state, even without repeating structural and functional testing with each visit. This approach can also be used in determining a course of treatment in order to prevent further progression of eye disease. While a lookup table is shown here, it is appreciated that this approach can be embodied entirely within the software instructions of a treatment system and is not required to be displayed. Further, it is appreciated that this example illustrates a relationship between two attributes of the IOP profile and that various other algorithms can be used to associate additional attributes.

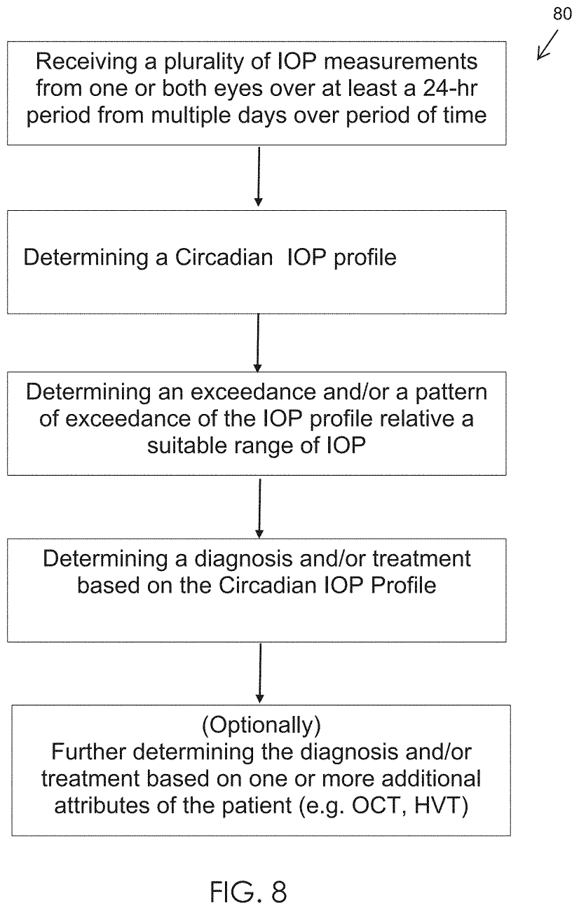

FIGS. 8-10 illustrate methods of assessing and treating a patient for eye disease. It is appreciated that the following methods are mere examples, and that such methods can further include additional steps or omit one or more steps described below, and still be in keeping with the concepts of the invention described herein.

FIG. 8 depicts method 80, which includes steps of: receiving a plurality of IOP measurements from one or both eyes over at least a 24-hr period from multiple days over period of time; determining a circadian IOP profile; determining an exceedance and/or a pattern of exceedances of the IOP profile relative a suitable range of IOP; and determining a diagnosis and/or treatment based on the circadian IOP Profile. These steps can be repeated during treatment and compared to the baseline IOP profile to evaluate efficacy of the treatment and/or to update the treatment as needed. The method can further include the step of determining the diagnosis and/or treatment based on one or more additional attributes of the patient (e.g., OCT, HVT).

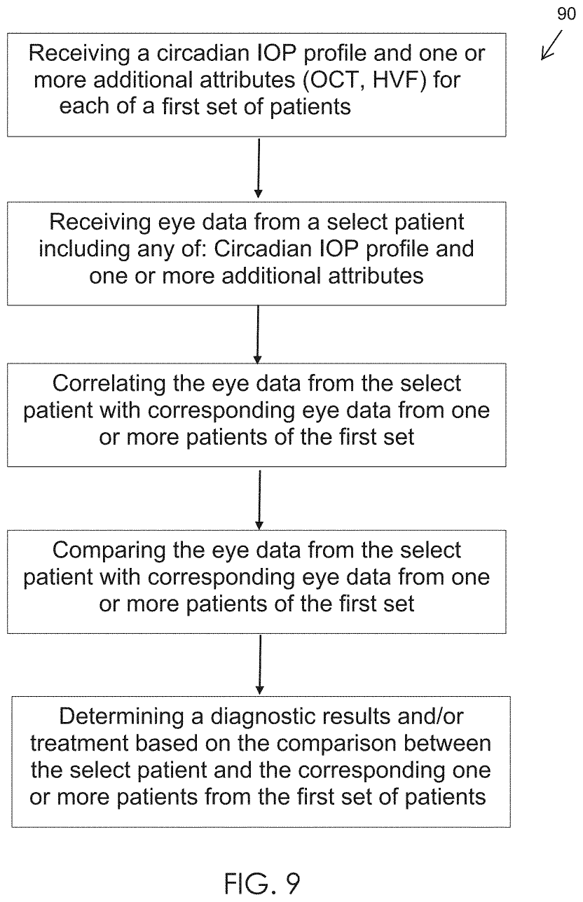

FIG. 9 depicts method 90, which includes steps of: receiving a circadian IOP profile and one or more additional attributes (e.g., OCT, HVF) for a first set of patients; receiving eye data from a select patient including any of: circadian IOP profile and the one or more additional attributes; correlating the eye data from the select patient with corresponding eye data from one or more patients of the first set; comparing the eye data from the select patient with corresponding eye data from one or more patients of the first set; and determining a diagnostic results and/or treatment based on the comparison between the select patient and the corresponding one or more patients from the first set of patients.

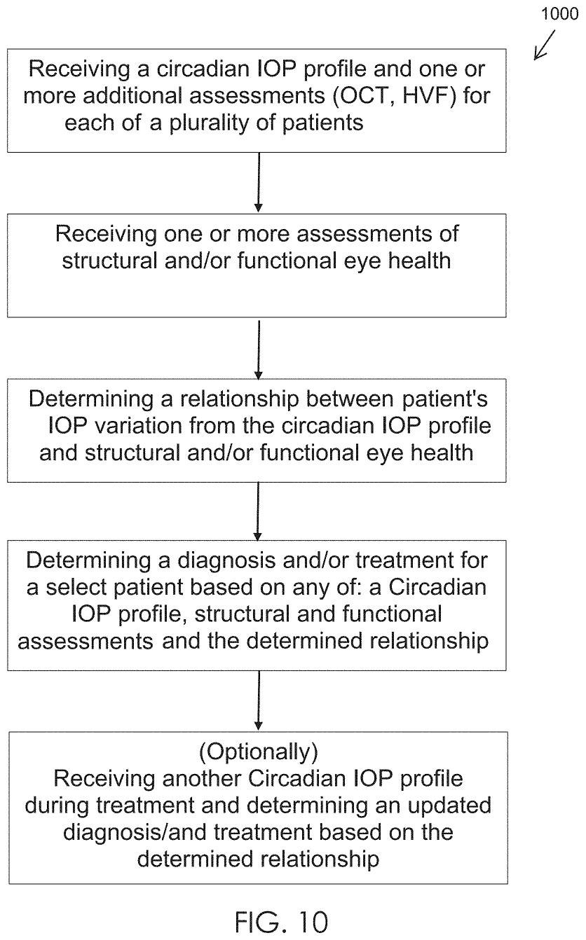

FIG. 10 depicts method 1000, which includes steps of: receiving a circadian IOP profile and one or more additional assessments (e.g., OCT, HVF) for a plurality of patients; receiving one or more assessments of structural and/or functional eye health; determining a relationship between a patient's IOP variation from the circadian IOP profile and structural and/or functional eye health; and determining a diagnosis and/or treatment for

the select patient based on any of: a circadian IOP profile, structural and functional assessments and the determined relationship. The method can further include a step of: receiving another circadian IOP profile during treatment and determining an updated diagnosis/and treatment based on the determined relationship.

In another aspect, after patterns of variation in IOP are identified and characterized, these patterns can be recognized from a collection or library of IOP data accumulated from multiple patients (e.g., by AI/DL). If the monitoring was done prior to the onset of the disease, this further allows for early detection and prevention by entering into a therapy that will maintain/shape the IOP profile towards the targeted IOP profile. It is recognized that IOP is defined as a risk factor and not as a diagnostic for glaucoma. The disease progression and understanding in conventional treatment approaches has significant gaps and lacking clear explanation into a multi-factorial paradigm that hasn't previously been modeled or quantified. One focus of the circadian rhythm plot of IOP is to represent and extract the dynamic state of the disease with IOP for primary open angle glaucoma (POAG) but in some cases, like normal tension glaucoma (NTG) (IOP<26 mmHg), these factors are more related to blood perfusion, oxygen saturation level and other hemodynamic factors (e.g., low blood pressure, oxidative stress due to large oxygen variation during sleep apnea). Thus, in some embodiments, other parameters (e.g., oxygen saturation level (oximetry) variation) could also be represented using such circular, circadian graph along with blood pressure, eyeball motion (REM) during sleep, blood pressure) to further inform diagnosis and treatment.

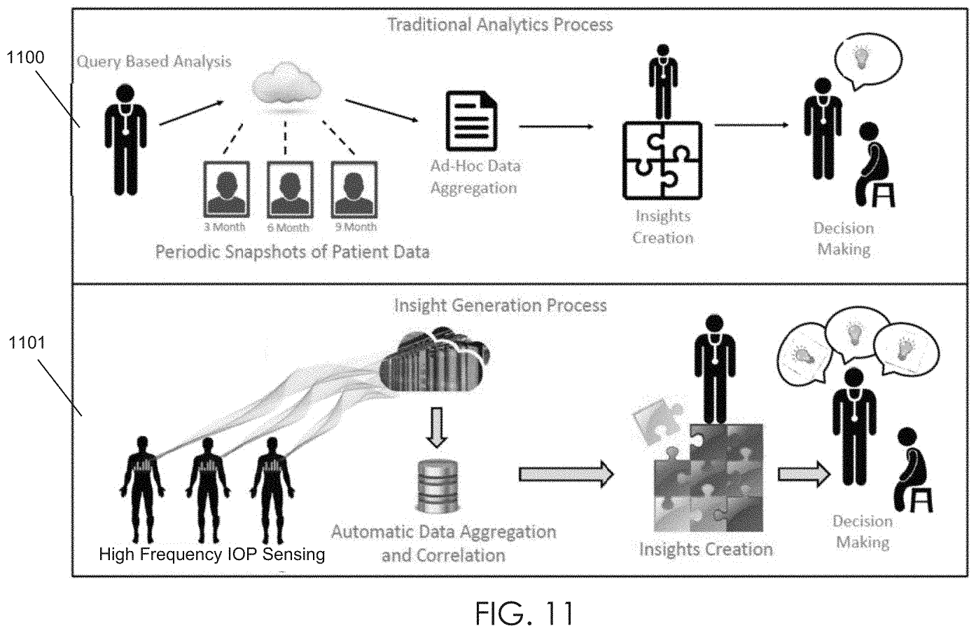

FIG. 11 illustrates the role of the graphical user interface within the decision process of the ophthalmologist by assessing more complex relationships that would be otherwise difficult or impossible to detect by looking at only a few data points. The traditional analytics process 1100 assesses a patient's condition with periodic snapshots, typically engaging in ad-hoc analysis and data integration, and determining a diagnosis or course of treatment with limited insight, which often has limited or unpredictable efficacy. The improved approach 1101 utilizing high frequency IOP sensing (e.g., circadian IOP profile with Targeted IOP profile) allows for automatic data aggregation and correlations, which allows insight from multiple relevant factors and similar patients from large patient populations, to facilitate improved decision making in both diagnosis and treatment options, thereby providing more patient-specific approach with an improved likelihood of efficacy. In this embodiment, a cloud-based approach can be used to connect patient-physician without the burden of additional office visits and only require an intervention if there is evidence of clinically actionable information.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

What is claimed is:

1. A method of diagnosing and/or treating an eye of a select patient:
receiving a first plurality of intraocular pressure (IOP) measurements over a first 24-hour period from the select patient;
determining a circadian IOP profile in a polar format based on the plurality of IOP measurements; and
displaying the circadian IOP profile on a graphical user interface.

2. The method of claim 1, further comprising:
receiving a second plurality of intraocular pressure measurements over a second 24-hour period;
wherein the circadian IOP profile is based on both the first and second plurality of IOP measurements.

3. The method of claim 1, further comprising:
displaying the circadian IOP profile on a graphical user interface along with an upper boundary and a lower boundary of suitable IOP for the select patient.

4. The method of claim 1, further comprising:
determining a pattern of exceedances of the circadian IOP profile from a suitable range of IOP, wherein the pattern of IOP exceedance comprises:
a number, a quantity, a timing, a total duration of exceedances, a maximum duration of individual exceedances or any combination thereof.

5. The method of claim 1, further comprising:
comparing the circadian IOP profile of the select patient with a plurality of circadian IOP profiles of a first set of patients; and
determining a risk assessment of the select patient and/or a course of treatment based on the comparison;
wherein comparing comprises comparing a pattern of IOP exceedances for the select patient with a library of patterns from a first set of patients.

6. The method of claim 5, wherein comparing further comprises comparing a baseline IOP pressure of the select patient with baseline IOP pressures of the first set of patients.

7. The method of claim 5, further comprising:
determining a relationship between the pattern of IOP exceedances and a structural and/or functional assessment from the first set of patients,
wherein the risk assessment and/or course of treatment is based on the relationship.

8. The method of claim 7, wherein the structural assessment comprises OCT; andwherein the functional assessment comprises HVF.

9. The method of claim 1, further comprising:
receiving a second plurality of IOP measurements from the select patient during treatment;
determining an updated circadian IOP profile from the second plurality of IOP measurements;
determining an updated pattern of IOP exceedances from the updated circadian IOP profile; and
determining an updated risk assessment and/or course of treatment based on the comparison.

10. The method of claim 1, wherein the plurality of IOP measurements are received from an implantable IOP sensor device having an IOP sensor portion that is disposed entirely within the vitreous body of the eye of the select patient.

11. A system for diagnosis and/or treatment of an eye of a select patient:
a graphical user interface; and
a processor having a memory with programmable instructions recorded thereon, the instructions configured to:
receive a first plurality of intraocular pressure (IOP) measurements over a first 24-hour period from the select patient;
output to the graphical user interface, a circadian IOP profile in a polar format based on the plurality of IOP measurements.

12. The system of claim 11, wherein the instructions are further configured to:
receive a second plurality of intraocular pressure measurements over a second 24-hour period;
wherein the circadian IOP profile is based on both the first and second plurality of IOP measurements.

13. The system of claim 11, wherein the instructions are further configured to display along with the circadian IOP profile an upper limit of suitable IOP for the select patient.

14. The system of claim 11, wherein the instructions are further configured to display along with the circadian IOP profile a lower limit of suitable IOP for the select patient.

15. The system of claim 11, wherein the instructions are further configured to determine a pattern of exceedances of the circadian IOP profile from a suitable range of IOP;
wherein the pattern of IOP exceedance comprises: a number, a quantity, a timing, a duration of exceedances, or any combination thereof.

16. The system of claim 11, wherein the instructions are further configured to:
compare the circadian IOP profile of the select patient with a plurality of circadian IOP profiles of a first set of patients; and
determine a risk assessment of the select patient based on the comparison and/or a course of treatment based on the comparison.

17. The system of claim 16, wherein comparing comprises comparing a pattern of IOP exceedances for the select patient with a library of patterns from a first set of patients.

18. The system of claim 16, wherein comparing further comprises comparing a baseline IOP pressure of the select patient with baseline IOP pressures of the first set of patients.

19. The system of claim 11, wherein the instructions further comprise:
receiving a second plurality of IOP measurements from the select patient during treatment;
determining an updated circadian IOP profile from the second plurality of IOP measurement;
determining an updated pattern of IOP exceedances from the updated circadian IOP profile; and
determining an updated risk assessment and/or course of treatment based on the comparison.

20. The system of claim 11, further comprising:
an implantable IOP sensor device having an IOP sensor portion that is disposed entirely within the vitreous body of the eye of the select patient.
